(19) United States
(12) Patent Application Publication
(10) Pub. No.: US 2024/0100215 A1
(43) Pub. Date: Mar. 28, 2024

(54) WEARABLE ULTRAVIOLET LIGHT SHIELD

(71) Applicant: Tyler SMITHSON, Huntington Beach, CA (US)

(72) Inventor: Tyler SMITHSON, Huntington Beach, CA (US)

(21) Appl. No.: 18/372,668

(22) Filed: Sep. 25, 2023

Related U.S. Application Data
(60) Provisional application No. 63/409,760, filed on Sep. 24, 2022.

(51) Int. Cl. A61L 9/20

(52) U.S. Cl. CPC A61L 9/20; A61L 2209/15

(57) ABSTRACT

A wearable protective device includes headwear having a brim and a germicidal light source attached to the brim. An optical reflective channel is designed to direct light generated by the germicidal light source away from the headwear and downward to illuminate area in front of a wearer's face. The headwear may be virtually any type of brimmed head garment. The germicidal light source is preferably an ultraviolet light source that generates UV-C light radiation to destroy airborne pathogens, including bacteria and viruses.

### RELATED APPLICATIONS

This application claims priority to U.S. Provisional Patent Application No. 63/409,760 filed Sep. 24, 2022, the entire contents of which are incorporated herein by reference.

### BACKGROUND OF THE INVENTION

### Field of the Invention

The invention disclosed herein relates generally to wearable protective equipment, more specifically to a wearable garment that offers protection against airborne pathogens, and most specifically to a wearable head garment that sterilizes air using ultraviolet light.

### Description of Related Art

The need has arisen to develop an alternative method for protecting individuals from the spread of viruses and bacteria. Many lives have been disrupted by the COVID-19 crisis. As a result, there is a desire to develop and deploy technology, products, and solutions that clean, sanitize, disinfect, or even sterilize air and vapor passing between people, without disrupting interpersonal communication, in ways that are not made possible by the current state of the art. Current solutions provide cloth masks that block a person's mouth, which are undesirable for a number of reasons—ineffective blockage of pathogens, disruption of verbal communication, discomfort, breathing impairment (especially during exercise), loss of facial communication through expression, fogging of eyeglasses, etc. Another prior art solution implemented by many businesses has been the use of clear, plastic barriers positioned between a worker and a customer. However, plastic barriers are similarly ineffective because air currents can pass around the barrier, and cause other discomforts such as stagnant air and sweating. Such barriers also impede communication between persons and present a failure potential if a barrier is impacted or falls down.

What is needed is a nondisruptive protective barrier that can be implemented by individuals and businesses alike to provide protection from microscopic airborne pathogens.

### SUMMARY OF THE INVENTION

The invention disclosed herein is a wearable garment that has germicidal properties to protect a user wearing the garment from airborne pathogens. The wearable protective device further protects others around the wearer from pathogens exhaled by the wearer that could be inhaled by others in the area.

In some embodiments, the wearable protective device has headwear with a brim. The headwear can be virtually any type of brimmed head garment, such as a baseball cap, bucket hat, cowboy hat, etc. A germicidal light source is attached to the brim of the headwear, preferably at the distal or forward most end of the brim, to focus light generated from the source on three dimensions with concentration and diffusion characteristics that put the most intense treatment areas at the breath intake and exhale zones of a user wearing the headwear while limiting any skin or eye exposure to the user. Further, an optical reflective channel is attached to the headwear and is designed to direct the light generated by the germicidal light source away from the headwear. The germicidal light source may be UVC, Far-UV with LED, fiber optic glow, or other directed arcing technologies.

In some embodiments, the germicidal light source is an ultraviolet light source. The germicidal light source generates a frequency or frequencies of light that will destroy airborne pathogens. Preferably, the ultraviolet light source generates ultraviolet-C wavelengths, which are known to be found in the 100 to 280 nanometer range of the electromagnetic spectrum. The germicidal light source is positioned and oriented so that light generated thereby is directed downward, and at optimal angled focusing formats, from the brim of the headwear. The optical reflective channel is further designed to deflect light generated by the germicidal light source away from a user's face when the user wears the device. In combination, the germicidal light source and the optical reflective channel are designed to direct and deflect the generated light down from the headwear and outward, away from the wearer's face and into a concentrated high intensity location where inhale and exhale vapor is most treatable.

Alternative embodiments of the protective device disclosed herein can have a plurality of germicidal light sources attached about the brim of the headwear. Preferably, each of the germicidal light sources is separated from an adjacent germicidal light source by a substantially equal distance. Light generated by each of the plurality of germicidal light sources is directed through the optical reflective channel and away from the headwear. Preferably, the light is directed from each germicidal light source in a common direction, which is preferably downward from the brim and away from a user's face when the user is wearing the device. Alternatively, the protective device may include a plurality of optical reflective channels so that each of the germicidal light sources are partially contained within one of the reflective channels. Thus, the invention may provide for one optical reflective channel per germicidal light source.

In some embodiments, the optical reflective channel is designed to be a double parabolic reflective channel. Alternatively, the optical reflective channel may have a closed end attached to the brim of the headwear and opposing reflective surface sidewalls extending therefrom. The total length of a first reflective sidewall is preferably greater than the total length of the second reflective sidewall. Further, the first reflective sidewall may also include an angled portion that is concave out. The angled portion of the first reflective sidewall is designed to ensure light is deflected away from the user's face when the user is wearing the device.

In some further alternative embodiments, a proximity detector may be attached proximate to the germicidal light source and in communication therewith. The proximity detector is configured to turn off the germicidal light source upon detection of an object within the vicinity of the light source. In some embodiments, a power source is also coupled to the germicidal light source and configured to provide power thereto. The power source may also be coupled to the proximity detector to provide power thereto as well.

### BRIEF DESCRIPTION OF THE DRAWINGS

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. Component parts shown in the drawings are not necessarily to scale, and may be exaggerated to better illustrate the important features of the invention. Dimensions shown are exemplary only. In the drawings, like reference numerals may designate like parts throughout the different views, wherein:

FIG. 1A is a side view of a user wearing one embodiment of a protective device according to the invention disclosed herein and FIG. 1B is a magnified side view of the optical reflective channel from the embodiment of the protective device of FIG. 1A.

FIG. 2 is a side view of a user wearing an embodiment of the protective device according to the present invention and a light intensity map positioned in front of the user.

FIG. 3 is a frontal view of a user wearing an embodiment of the protective device according to the present invention and having a light intensity superimposed over the user's face.

FIGS. 4A to 4D are various light intensity maps exemplifying various intensity profiles that may be generated by one or more embodiments of the protective device according to the present invention.

FIGS. 5A to 5C are various other light intensity maps exemplifying various intensity profiles that may be generated by one or more embodiments of the protective device according to the present invention.

FIG. 6A is a side view of an alternative embodiment of a protective helmet according to the present invention and FIG. 6B is a magnified side view of the double reflective channel from the embodiment of the protective helmet of FIG. 6A.

### DETAILED DESCRIPTION OF THE INVENTION

The following disclosure presents exemplary embodiments of garments, such as hats, visors, or other wearable headgear, that possess germicidal properties to protect a user who is wearing the garment from airborne microscopic particles and pathogens. The germicidal properties protect the wearer from microscopic bacteria, viruses and other pathogens that are commonly suspended in water droplets in the air and could be potentially inhaled unknowingly by the wearer. Additionally, the disclosed protective devices will provide a layer of protection for individuals in the vicinity of the wearer. The protective device not only deploys the germicidal properties against incoming particles but also against outgoing or exhaled particles. This is particularly useful for persons that may currently be ill with an infectious pathogen, such as the Covid-19 virus, but also for healthy persons who may be closely associated with ill or immunocompromised persons, such as persons working in a hospital or nursing home setting.

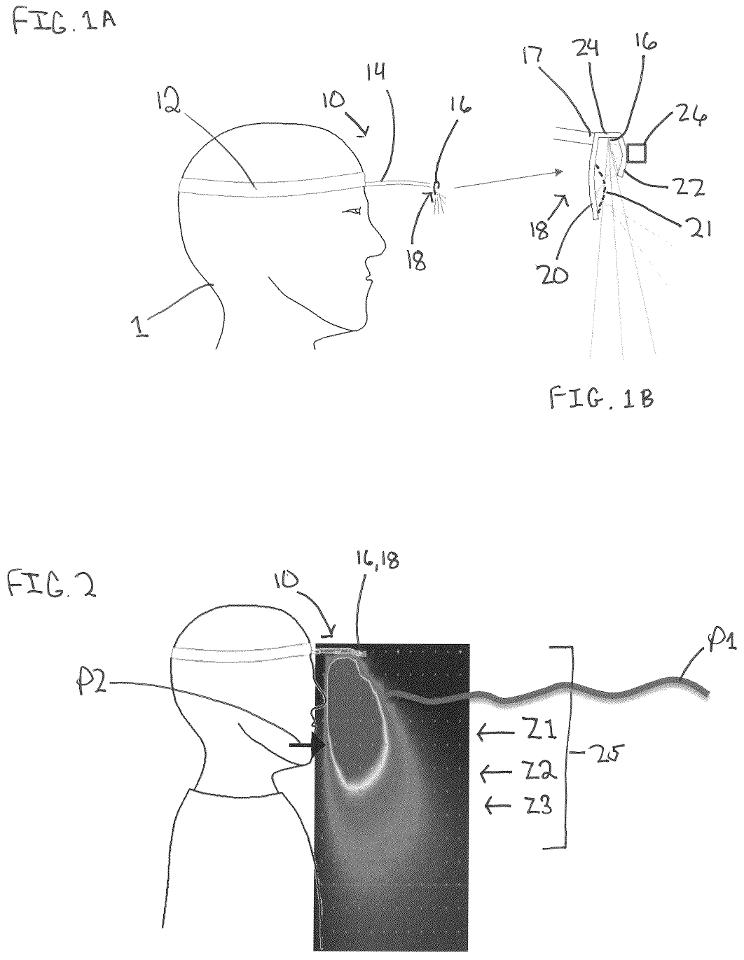

FIG. 1A is a side view of a user wearing an embodiment of a protective device 10 according to the present invention. The protective device 10 includes a hat 12 with a brim 14 extending therefrom. Note, Applicant may use the word “hat” interchangeably herein with words such as headwear, hard hat, baseball cap, helmet, etc. with the understanding that any of these words used in the context of this application refer, generally, to wearable head garments. The invention disclosed herein is not limited to the specific type of wearable head garment and can be used with virtually any type of garment a person may wear on their head, including, hats, hardhats, baseball caps, helmets, medical shields, cowboy hats, bucket hats, etc.

The protective device 10 includes at least one germicidal light source 16 attached to the brim 14. Preferably, the germicidal light source 16 is attached to the distal end 17 of the brim 14, which is understood to mean the edge of the brim that extends forward and away from the wearer's face. In some embodiments, such as the one illustrated in FIG. 1A, there may be two or more germicidal light sources 16 attached to the distal end 17 of the brim 14. The germicidal light source 16 is preferably an ultraviolet light source that emits light in the ultraviolet-C wavelength (hereinafter, “UV-C radiation” or “UV-C light”), which are found in the 100 to 280 nanometer range of the electromagnetic spectrum. UV-C light is preferred as the germicidal light source 16 because such light is known to disrupt the DNA and RNA of bacteria, viruses and other types of pathogens thereby destroying the pathogen by preventing replication.

The germicidal light source 16 illuminates the area immediately in front of a user 1 who is wearing the device 10. FIG. 1B is a magnified side view of the distal end 17 of the protective device 10 illustrated in FIG. 1A. To ensure the germicidal light source 16 does not illuminate the face of the user 1 when wearing the device 10, an optical reflective channel 18 is provided to partially contain the light source therein. The optical reflective channel 18 is similarly attached to a distal end 17 of the brim 14. In some embodiments, the optical reflective channel 18 may be configured as a double parabolic channel with a first parabolic reflective surface 20 opposing a second parabolic reflective surface 22.

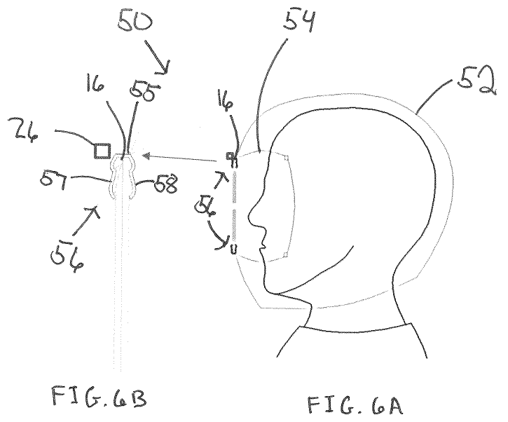

In some embodiments, the optical reflective channel 18 has a closed end 24 attached to the brim 14 and at least two opposing reflective surfaces 20, 22 extending therefrom. The germicidal light source 16 is preferably attached at the closed end 24 between the two opposing reflective surfaces 20, 22. In some embodiments, the total length of the first reflective surface 20 is greater than the total length of the second reflective surface 22. The first reflective surface 20 may also include an angled portion 21 that is concave out. In other words, the angled portion 21 of the first reflective surface 20 preferably curves away from the brim 14 in a direction toward the second reflective surface 22. The sharpness of the curve for the angled portion 21 may be used to control the intensity of the UV-C radiation proximate to the face of the user 1, as discussed in more detail with regard to FIGS. 5A-5C.

In some embodiments, a proximity detector 26 may be included with the protective device 10. The proximity detector 26 is positioned proximate to the germicidal light source 16. The proximity detector 26 is configured to be in communication with the germicidal light source 16. In preferred embodiments, the proximity detector 26 is configured to automatically turn off the germicidal light source 16 in response to the detector 26 detecting an object in close proximity to the light source. Thus, the proximity detector 26 is implemented as a redundant safety system, in conjunction with the optical reflective channel 18, for the protective device 10 to ensure minimal exposure to ultraviolet radiation for a user. For instance, when a user 1 wears the protective device 10 while eating, the proximity detector 26 can automatically shutoff the germicidal light source 16 whenever the user raises their hand to their mouth to limit ultraviolet radiation exposure to their hand.

The protective device 10 further includes a power source electrically coupled to the germicidal light source 16. The power source is configured to provide power to the germicidal light source 16. In some embodiments, the power source may be a replaceable or rechargeable battery or battery pack embedded in the brim 14 or otherwise attached to the hat 12. The power source can also be electrically coupled to the proximity detector 26 and is configured to provide power thereto. In a more elaborate embodiment, the garment may further comprise a means for charging the power source, such as one or more photovoltaic cells mounted atop the garment, for example, on the visor of a baseball cap, that are electrically coupled to the rechargeable battery.

FIG. 2 is a side view of a user wearing an embodiment of the protective device 10 and a light intensity map positioned in front of the user's face. The germicidal light source 16 generates three different zones of radiation intensity to define an intensity profile 25. The first zone Z1 has the highest intensity of UV-C radiation and is largest of the three radiation zones. The intensity of the germicidal light source 16 and the configuration of the optical reflective channel 18 is therefore selected to ensure that radiation zone Z1 is large enough to substantially cover the area in the immediate vicinity of a user's face when wearing the device 10. Thus, in some preferred embodiments, the germicidal light source 16 has the power to illuminate an area of roughly 10 to 20 inches vertically from the light source and roughly 5 to 10 inches horizontally from the light source. When a user wears the device, this would be about 10 to 20 inches downward from the distal end 17 of the brim 14 and about 5 to 10 inches outward from the brim, away from the user's face, caused by the reflective surfaces 20, 22 of the optical reflective channel 18.

As the UV-C radiation from the germicidal light source 16 reaches the end of the first radiation zone Z1, it begins to dissipate and a second radiation zone Z2 can be defined. Similarly, a third radiation zone Z3 is defined below the radiation zone Z2 and has the lowest intensity of UV-C radiation.

According to the invention, the first radiation zone Z1 is effective at destroying pathogens P passing therethrough. Zone Z1 when illuminated with UV-C radiation for a sufficient exposure time is up to 99% effective at destroying the RNA structure of viruses, to effectively disable pathogens P1, such as viruses, and prevent them from spreading and causing harm. The UV-C radiation in Z1 also kills bacteria to similarly prevent harm and spread. Pathogens P1 may be airborne pathogens exhaled by an individual in the vicinity of the user. The drop in radiation intensity at zone Z2 results in the UV-C radiation being up to 95% effective at eliminating incoming P1 and outgoing P2 pathogens. Zone Z3 has the lowest efficacy rate of up to 90%, resulting from a further drop in intensity of the UV-C radiation illuminated from the germicidal light source 16. However, since zone Z3 is the most distal from a user's face when wearing the protective device 10, the drop in efficacy of the UV-C radiation generated by the germicidal light source 16 is acceptable as any surviving pathogens would not be directly inhaled by the user.

FIG. 3 is a frontal view of a user wearing an embodiment of the protective device 10, according to the present disclosure, with a light intensity map superimposed over the user's face. To ensure zone Z1 can cover the width of a user's face, more than one germicidal light source 16 may be positioned about the brim 14 of the hat 12. In the illustrated embodiment, there are two germicidal light sources 16 positioned apart from one another about the distal end 17 of the brim 14. In some preferred embodiments, each germicidal light source 16 is partially contained within an optical reflective channel 18 so that there are an equal number of light sources and reflective channels. In alternative embodiments not shown, the optical reflective channel may extend the length of the distal end 17 of the brim 14 so that a single, stretched channel can be used to deflect UV-C radiation from multiple germicidal light sources 16 positioned about the brim. The number and positioning of the germicidal light source 16 about the brim 14 may be dependent on the type of hat 12 and further dependent on the intensity of the light source, where higher intensity light sources may require a fewer total number about the brim.

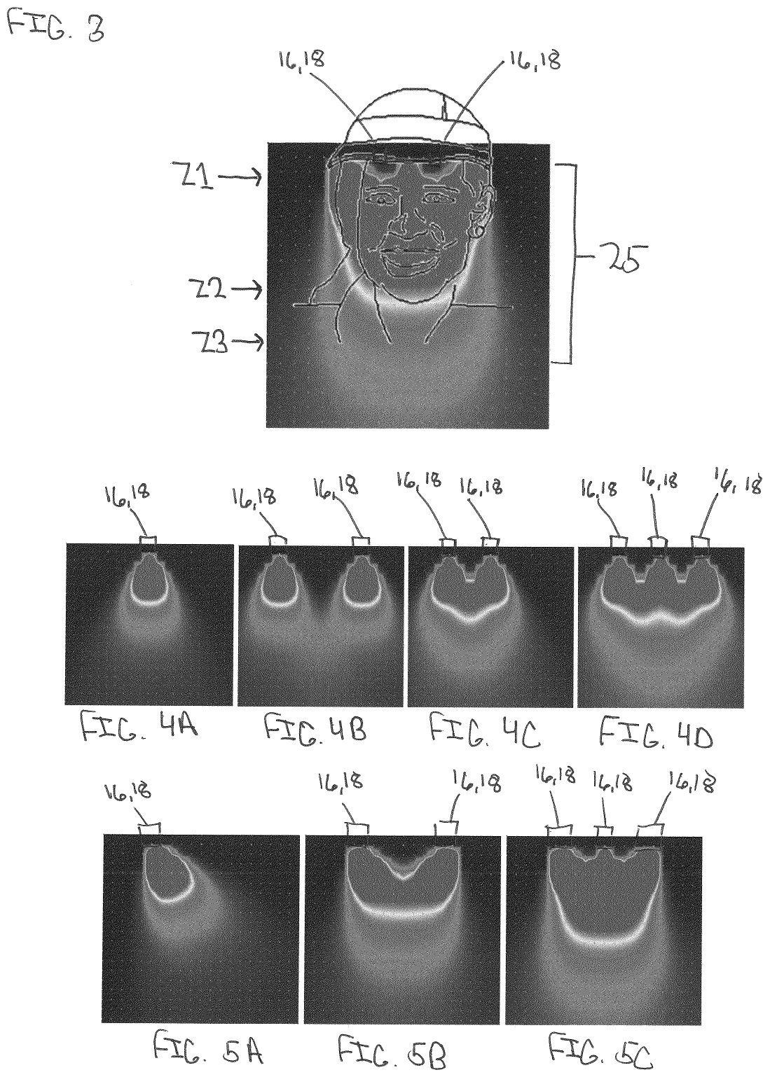

FIGS. 4A to 4D are examples of different light intensity maps that can result from various embodiments of a protective device 10 according to the present disclosure. The difference between the light intensity maps of FIGS. 4A to 4D are a result of a different number and positioning of germicidal light sources 16 within one or more optical reflective channels 18 about the brim 14. For example, FIG. 4A exemplifies the varying levels of UV-C intensity caused by one germicidal light source 16 within one optical reflective channel 18 and positioned at a substantially center point of the distal end 17 of the brim 14. In FIG. 4B, there are two germicidal light sources 16 each contained within an optical reflective channel 18 and positioned about opposing edges of the distal end 17 of the brim. In contrast, FIG. 4C illustrates the intensity of UV-C radiation resulting from two germicidal light sources 16 each partially within an optical reflective channel 18 and positioned closer together on opposing sides of the distal center point of the brim 14. Further, the UV-C intensity illustrated in FIG. 4D can be a result of three germicidal light sources 16 each partially contained within an optical reflective channel 18 and positioned an equal distance from one another about the distal end 17 of the brim 14. Many other forms of the intensity profile resulting from the protective device 10 may be possible and are dependent on the relative number and positioning of the germicidal light sources 16 about the brim 14.

In addition to the number and positioning of the germicidal light sources 16 about the brim 14 impacting the intensity profile 25 for the UV-C radiation, the degree of curvature for the angled portion 21 of the optical reflective channel 18 can cause changes to the intensity profile. FIGS. 5A to 5C are examples of different light intensity maps that can be the result of various embodiments of the protective device 10 according to the present disclosure. Each of the intensity profiles 25 illustrated among FIGS. 5A to 5C result from a different degree of curvature for the angled portion 21 of the optical reflective channel 18. For example, the angled portion 21 in FIG. 5A may be considered to have a sharp curve so that a majority of the UV-C radiation from the germicidal light source 16 is deflected away from a user wearing the device 10. In contrast, the angled portion 21 for FIG. 5C may be described as significantly less sharp so that less UV-C radiation is deflected away from the user and is allowed to extend zone Z1 vertically below the brim 14. FIG. 5B is an example of an intermediate degree of curvature for the angled portion 21 of the optical reflective channel 18. In FIG. 5C, for example, the effective shape of the light profile is generated by configuring the light sources at particular angles.

FIG. 6A is a side view of an alternative embodiment of a protective helmet 50 according to the present disclosure. FIG. 6B is a magnified side view of a portion of a double reflective channel deployed with embodiments of the protective helmet 50 as shown in FIG. 6A. The protective helmet 50 can be implemented with a variety of helmet or hood types, including but not limited to: football helmets, bicycle or skating helmets, snowboard or ski helmets, motorcycle helmets, hazmat hoods, etc. The protective helmet 50 includes a hood or helmet 52. The face opening 54 of the helmet 52 includes one or more germicidal light sources 16 partially contained within one or more double reflective channels 56. The double reflective channel 56 includes a closed end 55 and opposing reflective walls 57, 58. In preferred embodiments, the opposing reflective walls 57, 58 are substantially equal in total length and are engineered to deflect the UV-C radiation substantially vertically from the germicidal light source 16. In some alternative embodiments, one or both of the opposing reflective surfaces 57, 58 may be configured with a parabolic design. Other types of mathematical curves may also form the one or both of reflective surfaces 57, 58, such as cubic, elliptical, sinusoidal, hyperbolic, etc.

A second double reflective channel 56 may be positioned substantially opposite the first double reflective channel so that deflected UV-C radiation from the first double reflective channel is bounced back thereto. In effect, the opposing double reflective channels 56 in the protective helmet 50 result in a UV-C sheet or wall of radiation substantially covering the entirety of the face opening 54. A proximity detector 26 may similarly be attached proximate to the germicidal light source 16. The proximity detector 26 of the protective helmet 50 is configured to turn off the germicidal light source 16 when an object is detected to have penetrated the UV-C radiation wall formed by the opposing double reflective channels 56.

In any of the foregoing embodiments, the germicidal light source may be a 265 nm LED. Other embodiments are possible that use alternative light sources. In one such alternative, a fiber optic glowing cable in a channel may be mounted on the brim to produce similar effects, and significantly reduce or avoid altogether the air gap between LEDs at the brim. The fiber optic cable would be configured to have vertical curvature optimized like the angles shown in FIG. 5C. In another alternative, the germicidal light source may comprise a Far-UV excimer style lamp configured along a specially shaped spline to achieve a desired illumination volume.

Exemplary embodiments of the invention have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A wearable pathogen protection device, comprising:
a headwear having a brim;
a germicidal light source attached to the brim; and
an optical reflective channel configured to direct light from the germicidal light source away from the headwear.

2. The device of claim 1, wherein the germicidal light source comprises an ultraviolet light source.

3. The device of claim 2, wherein the ultraviolet light source generates ultraviolet-C wavelengths.

4. The device of claim 1, wherein the germicidal light source directs the light downward from the brim.

5. The device of claim 1, wherein the reflective channel is configured to deflect the light away from a user's face when the user wears the device.

6. The device of claim 1, wherein the reflective channel comprises a double parabolic reflective channel.

7. The device of claim 1, further comprising a proximity detector in communication with the germicidal light source.

8. The device of claim 7, wherein the proximity detector is configured to turn off the germicidal light source upon detection by the proximity detector of an object in close proximity to the germicidal light source.

9. The device of claim 1, further comprising a power source, wherein the power source is electrically coupled to the germicidal light source and is configured to power the germicidal light source.

10. The device of claim 1, further comprising a plurality of germicidal light sources.

11. The device of claim 10, wherein each of the plurality of germicidal light sources is positioned a substantially equal distance from an adjacent one of the germicidal light sources.

12. The device of claim 10, wherein light generated by each of the germicidal light sources is directed through the optical reflective channel.

13. The device of claim 10, further comprising a plurality of optical reflective channels, wherein each germicidal light source is partially contained within one of the plurality of optical reflective channels.

14. The device of claim 10, wherein the optical reflective channel is configured to deflect the light from each of the germicidal light sources in a common direction.

15. The device of claim 14, wherein the common direction comprises a direction downward from the brim and away from a user's face when a user wears the device.

16. The device of claim 1, wherein the reflective channel comprises a closed end attached to the brim and a first reflective surface and a second reflective surface extending from opposing ends of the closed end.

17. The device of claim 16, wherein the first reflective surface has a total length greater than a total length of the second reflective surface.

18. The device of claim 16, wherein the first reflective surface comprises an angled portion configured to deflect the light away from a user's face when the user wears the device.

19. The device of claim 18, wherein the angled portion of the first reflective surface is concave out.

20. The device of claim 1, wherein the germicidal light source is configured to destroy airborne microscopic organisms.
